(19) United States
(12) Patent Application Publication
(10) Pub. No.: US 2006/0045890 A1
(43) Pub. Date: Mar. 2, 2006

(54) Topical skin care compositions

(76) Inventors: Anthony D. Gonzalez, Oak Ridge, NJ (US); Barbara J. Hubbard, New Milford, NJ (US); Glen Anderson, Pleasantville, NY (US); Robert E. Kalafsky, Ogensberg, NJ (US)

Correspondence Address: Charles J. Zeller, Esq.;Avon Products Inc., Avon Place, Suffern, NY, 10901, US

(21) Appl. No.: 10/928,823

(22) Filed: Aug. 27, 2004

(51) Int. Cl. A61K 8/893

(52) U.S. Cl. 424/400; 424/70.12

(57) ABSTRACT

A novel combination of silicon resin, amorphous silica and low oil absorbing spherical powder is described which when added to topical skin care compositions containing high concentrations of active agents, improves their aesthetic feel and ease of application.

### FIELD OF THE INVENTION

The present invention relates to compositions topically applied to the human body, especially skin. More specifically, the present invention relates to compositions and methods for improving the aesthetic properties of topical compositions.

### BACKGROUND OF THE INVENTION

Topically applied skin care compositions are used to provide a variety of benefits. Cosmetic and personal care hygiene benefits include preventing exposure to harmful UV radiation in sunlight, reducing or preventing fine lines and wrinkles, improving the aesthetic appearance of the skin, providing moisturization to the skin, providing insect repellency to the skin, reducing pigmentation of the skin, protecting the skin from oxidation, wind and cold, treating diaper rash, exfoliating the skin, providing a cosmetic ingredient to the skin, and providing an antiperspirant or deodorant to the skin. Over the counter and prescription drug benefits include providing an antibiotic, antifungal, or antiviral product typically to the skin, and treating a particular disease condition of skin, such as acne or psoriasis, with a topical lotion, cream or ointment product.

In order to accomplish these functions, the skin care compositions contain a variety of active agents such as sunscreens, moisturizers, antioxidants, insect repellents, antiperspirants, topical analgesics, deodorants, antibiotics, antimicrobials, antifungals, anti-acne agents, and skin protectants. These active agents need to be present at a variety of concentrations in order to provide a defined level of functionality. In some situations it is necessary that the skin care composition contain high concentrations of one or more active agents, for example, a high concentration of sunscreen in a skin care composition to obtain high SPF protection from long term exposure to sunlight. In these situations the concentration of the active agent can result in the skin care product having an unpleasant aesthetic feel when applied to the skin. In other instances an active agent that is otherwise not unpleasant is provided in a composition that further contains an active agent or other component that results in an unpleasant aesthetic feel. Negative aesthetics thus may be attributable to a component of the vehicle, to a particular combination of vehicle components, to a particular active component and a vehicle component, or to a combination of active components.

If the skin care product is difficult to apply or is otherwise unpleasant to use it will increase the likelihood that the user will not comply with the usage protocol, or will cease use completely, and the beneficial effects of the product will not be realized. Depending on the nature of the product, this can prolong or exacerbate skin conditions such as photoaging, diaper rash, dry skin, eczema, and dermatitis. In some instances, nonuse or reduced use may result in not preventing a condition, for example the nonuse of a sunscreen or an insect repellent. Nonuse of a moisturizer can lead to dry skin.

Accordingly, it is desirable to reduce unpleasant tactile or sensory responses and generally improves the aesthetics of a variety of products, such as personal care, cosmetic, and pharmaceutical products.

It is therefore an object of the present invention to provide an aesthetic modifier for incorporation into products intended for topical application to the human body which improve topical application properties and aesthetic feel of the composition.

It is another object of the invention to provide an aesthetic modifier for compositions containing an active agent that would, but for the incorporation of the aesthetic modifier component, compromise the topical application properties and aesthetic feel of the composition.

A collateral aspect of the invention is to improve consumer compliance with product protocols by providing compositions with desirable topical application properties and aesthetic feel.

### SUMMARY OF THE INVENTION

It has been found that the aesthetic properties of a cosmetic, personal care/hygiene, or pharmaceutical product intended for topical application to the human body, especially skin, can be improved by the incorporation of a novel aesthetic modifier composition whereby compliance with prescribed usage is facilitated. The aesthetic modifier composition comprises a mixture of amorphous silica, silicone resin and a low oil absorbing spherical powder that provides unexpected performance compared to the individual properties of each component. The aesthetic modifier composition of the present invention is useful for incorporation in compositions that are applied topically. In another aspect of the invention the topical compositions contain an active agent present in an amount effective to achieve its intended benefit, as hereinafter described. The presence of aesthetic modifier compositions in such topical compositions ameliorates the deleterious effects of the active agents on the tactile properties of the topical composition.

The novel combination allows for maximum aesthetic enhancing benefits of each of the components while decreasing their negative attributes if they were used alone. For example, 1) Amorphous silica works well to degrease formulations, but it can make formulations draggy at effective levels. Whe used in combination with a low oil absorbing powder and silicone resin the degreasing effects are observed without the drag. 2) Silicone resins leave a silky film on the skin when dried. However, they go through a tacky phase during dry down. The aforementioned combination minimizes this tackiness, thus providing the end benefit of a silicone resin without the negative aesthetics experienced during application.

The unexpected beneficial characteristics of the three ingredients used in combination has, until now, been unknown. By using these three components in the novel combination described in the present invention, the positive properties of non-greasiness and smooth feel are maintained, while the negative properties of the additives such as drag and tackiness are reduced or eliminated. The present invention, by providing improved aesthetic feel, will increase the probability that the topical composition will be applied as intended and the beneficial effects of the product realized.

The present invention further provides a topical skin care formulation comprising at least one silicone resin, at least one amorphous silica, at least one low oil absorbing spherical powder and one or more active agents in a cosmetically acceptable vehicle. In one embodiment of the invention, silicone resin, amorphous silica and the low oil absorbing spherical powder are present in a ratio of about 1.0:0.01-2,000:0.05-5,000 on a weight to weight basis.

In a further embodiment of the invention, silicone resin, amorphous silica and the low oil absorbing spherical powder are present in a ratio of about 1.0:0.07-100:0.7-300 on a weight to weight basis in the skin care composition.

In a more preferred embodiment, silicone resin, amorphous silica and the low oil absorbing spherical powder are present in a ratio of 1.0:1.0-10:2.0-20 on a weight to weight basis in the skin care composition.

In preferred embodiments of the invention the oil absorption capacity of the spherical powder is less than about 250%, based on the weight of the spherical powder. That is, the spherical powder preferably absorbs less than about 2.5 times its own weight of oil. Typically, the oil absorption capacity is between about 50% by weight and about 250% by weight. For more preferred embodiments the oil absorption is about 150% or less by weight of the spherical powder.

Another embodiment of the invention provides a sunscreen composition. The use in combination of amorphous silica, silicone resin and low oil absorbing spherical powder allows high concentrations of sunscreen agents to be used while still maintaining a composition that is pleasant to apply and has a pleasing aesthetic feel on the skin.

A further embodiment of the invention provides for an over the counter skin protectant such as for example, an insect repellent product, comprising amorphous silica, silicone resin and low oil absorbing spherical powder, which is easy to apply and has a pleasant feel on the skin.

### DETAILED DESCRIPTION OF THE INVENTION

As used herein the terms of art are intended to their ordinary and accustomed meaning unless otherwise indicated. All concentrations are provided as percent by weight of the entire composition, unless otherwise indicated. All ratios are provided on a weight basis unless otherwise indicated. Concentrations are “as is” unless indicated to the contrary.

The term “aesthetic modifier composition” refers to the inventive combination of amorphous silica, silicone resin, and spherical powder. The aesthetic modifier composition improves the aesthetics of topical compositions of the present invention that are intended for topical application to the human body.

By the term “active agent” we mean any ingredient for use in topical compositions that provide a benefit to the skin and is not present merely as an inert substance used as a diluent or vehicle adjuvant. Cosmetic active agents are agents that provide a cosmetic benefit to the skin that is, improving the appearance of skin, protecting the skin, maintaining the skin in good condition, or applying a personal hygiene product to the skin. Pharmaceutic agents may be over the counter and prescription, and provide a benefit in the treatment of a medical condition or disease.

Topical compositions are compositions intended for application to the human body, and in particular for application to skin. Skin means human skin, lips and nails.

The present invention relates to the use of a novel combination of silicone resin, amorphous silica and low oil absorbing spherical powder in topical skin care products to modify the aesthetics of a topical composition. It has been found surprisingly that the use of these three ingredients in combination improves the tactile feel of topical skin treatment products. Many such products supply vehicles that have undesireable tactile properties, such as oiliness. Further, certain skin treatment products that have a concentration of one or more active agents in an amount that typically results in poor tactile aesthetics, such as sunscreens, have greatly improved aesthetic feel when this combination of ingredients is added to the product.

Preferred topical compositions for use with the inventive concepts are those containing at least one ingredient that imparts to a conventional skin care composition an unattractive aesthetic feel. This unattractive feel is often deemed unacceptable to the user of a consumer product and disliked by the user of a drug product, and often results in insufficient consumer use as defined by the prescribed usage protocol or even nonuse.

These unattractive properties are most often present when the active agent is a lipophilic compound, and can exist even when the lipophilic compound is present in low concentration. Moreover, these unattractive properties can also be present when the active agent is a hydrophilic compound, especially when the hydrophilic compound is present in the composition at high concentration. Additionally, the unattractive tactile properties of the conventional composition may be attributable to one or more adjuvants or vehicles present in the conventional composition.

The unattractiveness of the conventional composition not containing the aesthetic modifier composition of the present invention is characterized by a greasy or oily feel, a definite tackiness when the composition on the skin is touched, a drag during application of the composition to the skin, the slow absorption of the composition into the skin even with extended rubbing of the composition into the skin, and heavy afterfeel. Beneficially, the aesthetic modifier composition of the present invention imparts exceptional tactile properties to such conventional compositions, substantially removing or mitigating their unattractive aesthetics.

1. Aesthetic Modifier Compositions

The aesthetic modifier compositions of the present invention comprise silicone resin, amorphous silica, and low oil absorbing spherical powder. Appropriate compositions for each of these components are provided below.

Silicone Resin

The silicone resins for use in the invention can be any organosilicone polymer wherein the pure resin is a solid at 25° C. and has a molecular weight from about 1,000 to about 10,000 daltons. The silicone resin is comprised of siloxane monomer units which may be monofunctional (“M”) units having the formula [R₃SiO_{0.5}]ₓ, difunctional (“D”) units having the formula [R₂SiO]_{Y}, trifunctional (“T”) units having the formula [RSiO_{1.5}]_{Z} and tetrafunctional (“Q”) units having the formula [SiO2]_{W} where R is preferably methyl but may also be, but is not limited to, C2 to C16 alkyls, vinyl, phenyl, amine or hydroxyl group. X, Y, Z and W can be any integer such that the molecular weight of the polymer is from about 1,000 to about 10,000 daltons. Formula I illustrates an unbranched organosilicone polymer, which are preferred.

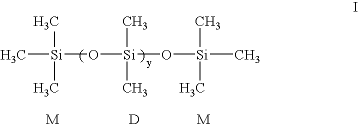

When “T” and “Q” units are used, branched polymers are possible such as the representative polymer illustrated in formula II. One skilled in the art will recognize that formula II illustrates only one example of the polymers that can be formed by mixtures of M, D, T and Q monomers. Further, it is known to those skilled in the art that the structure of the polymer can be influenced by adjusting the ratios of the M, D, T and Q polymers.

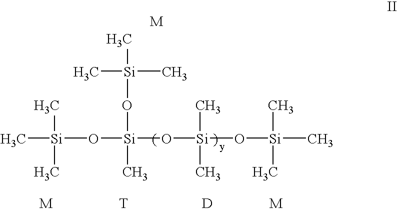

A non-limiting example of a suitable silicone resin is trimethylsiloxysilicate. Silicone resins are widely available from commercial sources and methods for their manufacture are well known in the art. Typical starting materials used in the manufacture of silicone resins include but are not limited to monomethyl-, dimethyl-, monophenyl-, diphenyl-, methylphenyl-, monovinyl- and methylvinyl chlorosilanes. A useful silicone resin is sold by Dow Corning under the trade name DC 593 Fluid (INCI Name: Dimethicone and Trimethylsiloxysilicate). Another suitable commercially available silicone resin is KF-7312 sold by Shin Etsu Chemical Company.

Amorphous Silica

Amorphous silica for use in the invention can be any particle composed of SiO₂ that has an irregular shape. Natural sources of amorphous silica include but are not limited to diatomaceous earth, quartz powder, sand and flint. In addition, amorphous silica can be manufactured by injecting chlorosilanes, such as silicone tetrachloride, into a flame of hydrogen and air. The resulting reaction produces hydrophobic fumed silica, which consists of very fine particles with high surface area. The amorphous silica typically has a surface area of from about 100 to about 400, preferably from about 150 to about 250 m² by gas absorption. This high surface area enables the amorphous silica to absorb a high amount of oil, often greater than 500%, preferably greater than 300%, and most preferably greater than 100% its own weight. Commercially available amorphous (or fumed) silicas are made by Cabot Corporation under the trade name Cabosil and by Degussa AG under the trade name Aerosil.

Spherical Powder

Low oil absorbing spherical powder is comprised of spherical particles made from a variety of materials including but not limited to silica, nylon, boron nitride, polymethylmethacrylate, ethyl acrylate, polytetrafluoroethylene (Teflon™), polyurethane powder, silicone rubber, talc, mica, serecite, polyvinyl alcohol, acrylates copoloymer and organomodified polysiloxane. Preferred spherical powders are made from silica, boron nitride or polyurethane powder. Suitable spherical powders are readily available from a variety of commercial sources.

The degree of oil absorption is typically less than about 250% by weight. That is, the spherical powder preferably absorbs less than about 2.5 times its own weight of oil. Typically, the oil absorption capacity is between about 50% by weight and about 250% by weight, and more preferably less than about 150% by weight.

The spherical powders are substantially spherical, that is, they may have surface anomalies or may be somewhat out of round owing to manufacturing variations or to minor fracturing of the powders during shipment. The powders have a diameter of from 1 to about 100 microns, preferably from about 5 to about 50 microns, most preferably from about 5 to about 25 microns.

Illustrative commercially available materials are Rubinate polyurethane powder available from Huntsman of Belgium; DC 9506 powder available from Dow Corning; BPA polymethylmethacrylate available from Kobo Products; and MSS-500N silica available from Kobo Products.

The silicone resin, amorphous silica and low oil absorbing spherical powder components of the aesthetic modifier composition are present in a preferred ratio relative to each other. In one preferred embodiment of the invention, silicone resin, amorphous silica and the low oil absorbing spherical powder are present in a ratio of 1.0:0.01-2,000:0.05-5,000 on a weight to weight basis. In a further more preferred embodiment of the invention, silicone resin, amorphous silica and the low oil absorbing spherical powder are present in a ratio of 1.0:0.007-100:0.7-300 on a weight to weight basis in the skin treatment composition. In a most preferred embodiment, silicone resin, amorphous silica and the low oil absorbing spherical powder are present in a ratio of 1.0:1.0-10:2.0-20 on a weight to weight basis in the skin treatment composition.

The weight ratios given herein are provided as the ratio of silicone resin to amorphous silica to low oil absorbing spherical powder. The weight ratios are normalized such that the silicone resin component is given as “1”. Where a range is recited, it will be understood that every value within the range is inherently disclosed. Thus, for example, a range of 0.1-2,000 discloses every value or sub-range therein.

2. Topical Compositions

The aesthetic modifier compositions of the invention are preferably employed in conjunction with compositions for topical application, such as to the hair nail or skin. In this regard, the aesthetic modifier composition may be added to any such product to improve its aesthetics.

In one non-limiting embodiment, topical compositions according to the invention are those comprising active agents or vehicles which exert deleterious effects on the aesthetics of the compositions in which they are contained. In this regard, the aesthetic modifier compositions are useful for imparting acceptable aesthetic properties to the vehicle.

Active Agents

Any active agent may be incorporated into the composition of the present invention. The active agents may be lipophilic or hydrophilic in character. The active agents may be oil soluble, oil insoluble, oil dispersible, water insoluble, water dispersible or water soluble. The unpleasant tactile feel as described above is sometimes characterized by the persistence of the composition on the skin for a period of time, that is, the slow absorption of the composition into the skin and/or the slow rate of evaporation from the skin.

As used herein, active agent includes cosmetic active agents used in the preparation of personal care/hygiene, over the counter, pharmaceutical or prescription pharmaceutical products. Cosmetic actives that exhibit these unwanted tactile properties include emollients that provide anti-oxidant, anti-wrinkle, moisturization, skin protection, sunscreens or ultraviolet light absorbers, and occlusives that retard evaporation of water from the skin.

Personal care/hygiene agents include antiperspirants and insect repellents.

Over the counter pharmaceuticals include over the counter antifungal agents, sunscreen, skin protectants, antimicrobial, anti-acne, antibiotic agents, antiviral agents, antibiotics, anti-fungal, psoriasis agents, topical analgesics and the like.

Sunscreen agents/ultraviolet light absorbers can be organic sunscreens, which include but are not limited to aminobenzoic acid (PABA), avobenzone, cinoxate, dioxybenzone, menthyl anthranilate, octocrylene, octyl methoxycinnamate, oxybenzone, padimate O, phenylbenzimidazole sulfonic acid, DEA methoxycinnamate, drometrizole trisiloxane, octyl salicylate, homomenthyl salicylate, octyl dimethyl PABA, TEA salicylate, 4-methyl benzylidene camphor, terephthalylidiene dicamphor sulfonic acid, ethyl PABA, hydroxy methylphenyl benzotriazole, methylene bis-benzotriazoyltetramethylbutylphenol, bis-ethylhexyloxyphenol methoxyphenol triazine, octyl triazone, polysilicone-15 (diethylbenzylidene malonate dimethicone) (sold as Parsol SLX by Roche), diethylhexyl butamido triazone (Uvasorb HEB sold by 3V, Inc.), diethylamino hydroxybenzoyl hexyl benzoate (Uvinul A Plus sold by BASF), zinc oxide, titanium dioxide or any derivatives, or any combinations thereof. Other useful sunscreen actives include those disclosed in U.S. Pat. Nos. 5,000,937 and 6,440,402, and in the International Cosmetic Ingredient Dictionary and Handbook, v. 4, Section 4, p. 2277-2278, 2294-2295 (9^{th} Edition, 2002) (hereinafter “INCI”), which sunscreen actives are incorporated herein by reference.

Moisturizers and skin protectants can be any of a wide variety of emollients and occlusive agents including oils, C12 to C24 fatty alcohols, esters of a fatty acids and an alcohol, glyceryl esters, lipids, ceramides, nonvolative silicone fluids, and the like. Suitable materials include but are not limited to lanolin, benzyl laurate, cetyl esters, dimethicone copolyol, glycerol isostearates, propyleneglycol laurate, glycerin, inulin, sorbitol, fish liver oil, squalane, methicone, mineral oil, petrolatum, allantoin, aluminum acetate, aluminum hydroxide, aluminum sulfate, calamine, cocoa butter, cod liver oil, colloidal oatmeal, dimethicone, glycerin, kaolin, lanolin, mineral oil, petrolatum, shark liver oil, sodium bicarbonate, natural oils such as jojoba oil and olive oil, essential oils, talc, witch hazel, zinc acetate, zinc carbonate and zinc oxide. Other suitable emollients, occlusives and skin protectants are found, for example, in 21 CFR §347 and 21 CFR §349 and in INCI, v. 4, Section 4, p. 2229-2252, incorporated herein by reference thereto.

Suitable antioxidants include but are not limited to ascorbic acid, caffeic acid, cysteine, hydroquinone and tocopherol.

Anti-acne agents include but are not limited to salicylic acid, benzoyl peroxide, resorcinol, and sulfur. See also INCI at p. 2883-2884 and 21 CFR §333.

Antimicrobial agents include but are not limited to alcohol, benzalkonium chloride, benzethonium chloride, hydrogen peroxide, methylbenzethonium chloride, phenol and povidone-iodine. Suitable antifungal agents include but are not limited to calcium undecylenate, povidone-iodine, undecylenic acid, tolnaftate and zinc undecylenate. See also INCI at p. 2282-2283. Insect repellents include any compound approved by the EPA for such use such as N,N-diethyl-meta-toluamide (DEET).

The active agent is present in the composition in an amount required to achieve its intended benefit, as is well known in the art. Generally, the active agent is present in an amount of less than about 50% by weight of the composition, although higher amounts are sometimes used, as in the case of a very high SPF sunscreen product. The incorporation of the aesthetic modifier composition permits incorporation of the active agent at high concentration levels, while maintaining attractive aesthetics of the composition.

In the case of emollients, occlusive agents and skin protectants, the amount present in the composition is typically from about 1 to about 35% by weight of the composition, preferably from about 2.5 to about 25%, most preferably from about 5 to about 20%. In the case of antioxidants, the amount present in the composition is from about 0.001 to about 5%, preferably from about 0.05 to about 1% by weight of the composition. In the case of anti-fungals the amount is from about 0.1 to about 5%, preferably from about 0.5 to about 2% by weight of the composition. In the case of sunscreen agents the amount is sufficient to obtain an SPF value of at least 2, preferably about 15 and above and most preferably about 30 and above. Very high SPF values, about 50 and above, are also contemplated. The amount of sunscreen actives present in the composition is typically from about 1 to about 40%, preferably from about 5 to about 25% by weight of the composition.

The topical compositions of the present invention may contain more than one active agent, for example an anti-fungal provided in a petrolatum base. Each active agent is present in an effective amount, with the total amount of actives generally being not more than about 75%, and typically not more than about 50% by weight of the composition, which concentration is achievable in a composition that has the improved aesthetics as previously described. The active agents may be lipophilic or nonlipophilic. The lipophilic active agent is generally immiscible in water, and typically has an insufficient number of polar groups to impart miscibility or even a moderate degree of dispersibility.

The nonlipophilic actives are often dispersible, miscible, or soluble in water. Such agents may exhibit some surface activity.

The insoluble active agents can be inorganic compounds such as zinc oxide. These active agents may also be organic compounds that provide a skin care benefit as described above.

The nonlipophilic actives can also adversely affect the aesthetic character of a composition, thereby requiring the incorporation of the aesthetic modifier composition of the present invention, especially when the nonlipophilic active is present. For example, although glycerin is hydrophilic, it is tacky and heavy when present in a skin care composition in an amount of about 10% by weight and above, particularly when present in an amount of about 20% by weight and above, and especially when present in an amount of about 30% by weight and above. Other nonlipophilic active agents for which the aesthetic modifier composition is particularly useful include coal tar, alpha-hydroxy acids and their salts, beta hydroxy acids, poly hydroxy acids, in particular salicylic acids, and their salts, kojic acid, hydroquinine, oxa acid and its salts, glyceryl polyacrylate, d-panthenol, and other polyols in addition to glycerin. In addition, the nonlipophilic actives are often incorporated in a composition that contains a lipophilic skin care active ingredient such as an emollient, occlusive, or skin protectant, e.g., topical zinc oxide in a petrolatum-containing composition.

The nonlipophilic active is present in the composition in an amount required to achieve its intended benefit, as is well known in the art. Analgesic actives such as benzocaine, dibucaine and lidocaine are present at about 0.5% to about 20% by weight. Some alcohol analgesics such as benzyl alcohol can be present at up to about 33% by weight. Dandruff and psoriasis actives such as coal tar and salicylic acid are present from about 0.1% to about 5% by weight. Alcohol antiseptics can be present from about 45% to about 95% by weight of the composition. Other antiseptics such as benzalkonium chloride and hexylresorcinol are present at about 0.1 to 1% by weight. Antiperspirant actives such as aluminum chlorohydrate, aluminum zirconium salts and related aluminum compounds are present at up to about 25% by weight. When used as a corn or callus remover salicylic acid can be present from about 12% to about 40% by weight of the composition. Anti-acne actives such as benzoyl peroxide, resorcinol, salicylic acid and sulfur can be present at about 0.5% to about 10% by weight.

The aesthetic modifier compositions of the invention are advantageously incorporated into any composition intended for topical application to skin, hair or nails, and is present in the topical composition in an amount effective to improve the aesthetic properties, especially the tactile feel, of the composition. Typically, the amount of the aesthetic additive blend in the composition is present in an amount of from about 0.1 to about 30% by weight of the composition, preferably from about 1 to about 20 wt. %, and most preferably from about 2 to about 10 wt. %.

Vehicle

The topical compositions of the present invention may contain a suitable vehicle or base for the active agents and the aesthetic modifier composition. However, in some topical compositions the aesthetic modifier composition may be suitable as the vehicle or base.

Cosmetically or pharmacologically suitable vehicle components include but are not limited to abrasive and particulates, inorganic and organic pigments, antifoam agents, buffering agents, chelating agents, fragrances and flavorants, plasticizers, polymers as film former, thickeners and surface active agents, surfactants as emulsifiers, hydrotropes, foam boosters, solublizing agents and suspending agents, viscosity modifying and control agents, preservatives, and solvents, especially C1 to C6 mono-, di- and polyhydroxyl alcohols, and water. The concentration of any particular constituent of the vehicle in the composition is the amount necessary to provide its intended function. Typically the vehicle comprises from about 0.1 to about 99%, preferably from about 1 to about 75%, most preferably from about 10 to about 50% by weight of the composition. Suitable adjuvants that make up the vehicle are identified, e.g., in Section 4 of the INCI reference.

3. Use of the Aesthetic Modifier Composition

The aesthetic modifier compositions of the invention are ideally suited for incorporation into products (i.e., compositions for topical application) having active ingredients which alter the aesthetics of the product. The aesthetic modifier compositions are useful to restore and/or improve the tactile characteristics of such products. This useful result is advantageously contemplated to assist in consumer compliance with a treatment regimen which would otherwise be compromised due to unsatisfactory consumer perception of a products aesthetics.

According to this aspect of the invention, the aesthetic modifier compositions are contemplated to be useful in products containing a sunscreen agent. In a preferred embodiment the sunscreen agent is present at a concentration sufficient to provide an SPF of 15 and above, more preferably an SPF of 30 and above, and most preferably an SPF of 50 and above, the SPF being determined by the method described in 21 CFR §352. In the case of even relatively low SPF products, the sunscreen agents are necessarily present in amounts that alter the aesthetic properties of the vehicle. The use of the aesthetic modifiers of the invention improves the feel of sunscreen products and likewise is predicted to increase the users frequency of application.

The aesthetic modifier compositions are useful in a variety of other products, including but not limited to, topical compositions for treating dry skin, topical compositions for preventing oxidation of the skin, topical compositions for treating microbial or fungal infections of the skin, topical compositions for treating acne, an topical compositions for protecting the skin from environmental insults.

In preferred embodiments the active ingredients, i.e., moisturizers, anti-oxidants, anti-fungals, antibacterials, anti-acne agents, etc., are present in the topical compositions.

As used herein, a “high” level of active ingredient indicates any concentration of active ingredient sufficient to alter the aesthetic, i.e. tactile, properties of the vehicle. For example, moisturizing products often contain upwards of 30% by weight glycerin as an active ingredient. At these levels, conventional moisturizers typically impart an oily, sticky or draggy feel to the skin due largely to glycerin rather than the vehicle. The affect of an active ingredient on the aesthetics of a product will of course depend on the nature of the active and therefore cannot be generally quantified. Actives which exert a pronounced effect on the aesthetics of the vehicle at much lower concentrations are also considered to be present in high concentration.

Compositions of the invention can be formulated by combining the silicone resin, amorphous silica and low oil absorbing spherical powder and adding the mixture to the composition in need of the enhanced aesthetic properties. Any of the standard methods known to those skilled in the art for formulating the products described above can be used in the preparation of compositions of the invention.

### EXAMPLES

Examples of illustrative compositions in accordance with the present invention are provided below.

### Example 1

### High SPF Sunscreen

| | Weight % |
|---|---|
| Demineralized Water | QS to 100 |
| Carbomer | 0.25 |
| Butylene Glycol | 3.0 |
| Preservative | 0.5 |
| Oxybenzone | 4.0 |
| Ethylhexyl-methoxycinnamate | 7.5 |
| Octyl Salicylate | 5.0 |
| Butyl Methoxydibenzoylmethane | 3.0 |
| Homomenthyl Salicylate | 8.0 |
| Emulsifier | 4.0 |
| Co-Emulsifier | 1.5 |
| Trimethylsiloxysilicate | 0.3 |
| Fumed Silica | 0.1 |
| Polymethylmethacrylate | 0.7 |
| Triethanolamine | 0.3 |

### Example 2

### Petrolatum Skin Protectant

| | Weight % |
|---|---|
| Demineralized Water | QS to 100 |
| Petrolatum | 30.0 |
| Emulsifier | 5.0 |
| Natural Lipids | 1.0 |
| Fumed Silicon Dioxide | 0.1 |
| Trimethylsiloxysilicate | 0.003 |
| Silica Spheres | 0.4 |
| Anionic Thickener | 0.28 |
| Preservative | 1.0 |
| Tromethamine | 0.3 |

### Example 3

### Insect Repellent

| | Weight % |
|---|---|
| Demineralized Water | QS to 100 |
| Carbomer | 0.25 |
| Butylene Glycol | 3.0 |
| Preservative | 0.5 |
| Oxybenzone | 4.0 |
| Ethylhexyl-methoxycinnamate | 7.5 |
| Octyl Salicylate | 5.0 |
| Butyl Methoxydibenzoylmethane | 3.0 |
| DEET | 10.0 |
| Emulsifier | 4.0 |
| Co-Emulsifier | 1.5 |
| Trimethylsiloxysilicate | 0.3 |
| Fumed Silica | 0.1 |
| Polymethylmethacrylate | 0.7 |
| Triethanolamine | 0.3 |

It should be understood that the foregoing description is only illustrative of the present invention. Various alternatives and modifications can be devised by those skilled in the art without departing from the invention. Accordingly, the present invention is intended to embrace all such alternatives, modifications and variations that fall within the scope of the appended claims.

What is claimed is:

1. A aesthetic modifier composition for modifying the aesthetics of a topical composition, said aesthetic modifier composition comprising:
a) silicone resin;
b) amorphous silica, and
c) low oil absorbing spherical powder.

2. The aesthetic modifier composition of claim 1 wherein the silicone resin, amorphous silica and the low oil absorbing spherical powder are present in a ratio to each other of 1.0:0.01-2,000:0.05-5,000 on a weight to weight basis.

3. The aesthetic modifier composition of claim 2 wherein the silicone resin, amorphous silica and the low oil absorbing spherical powder are present in a ratio to each other of 1.0:0.007-100:0.7-300 on a weight to weight basis.

4. The aesthetic modifier composition of claim 3 wherein the silicone resin, amorphous silica and the low oil absorbing spherical powder are present in a ratio to each other of 1.0:1.0-10:2.0-20 on a weight to weight basis.

5. The aesthetic modifier composition of claim 1 wherein the oil absorption capacity of the low oil absorbing spherical powder is less than about 2.5 times the weight of the spherical powder.

6. The aesthetic modifier composition of claim 5 wherein the oil absorption capacity of the low oil absorbing spherical powder is less than about 1.5 times the weight of the spherical powder.

7. The aesthetic modifier composition of claim 1 wherein the silicone resin is an organosilicone polymer comprising a plurality of siloxane monomer units selected from the group consisting of [R₃SiO_{1/2}]_{X}, [R₂SiO]_{y}, [RSiO_{1.5}]_{Z} and [SiO₂]W; wherein R is selected from the group consisting of methyl, C2 to C16 alkyl, vinyl, phenyl, amine and hydroxyl and x, y, z and w are integers selected such that the molecular weight of the polymer is from about 1,000 Daltons to about 10,000 Daltons.

8. The aesthetic modifier composition of claim 7 wherein R is methyl.

9. The aesthetic modifier composition of claim 8 wherein the silicone resin is trimethylsiloxy silicate.

10. The aesthetic modifier composition of claim 1 wherein the amorphous silica is selected from the group consisting of diatomaceous earth, quartz powder, sand, flint and fumed silica.

11. The aesthetic modifier composition of claim 1 wherein the low oil absorbing spherical powder is selected from the group consisting of silica, nylon, boron nitride, polymethylmethacrylate, ethyl acrylate, polytetrafluoroethylene, polyurethane powder, silicone rubber, talc, mica, serecite, polyvinyl alcohol, acrylates copoloymer and organomodified polysiloxane.

12. A composition for topical application to skin comprising the aesthetic modifier composition of claim 1.

13. The composition for topical application to skin of claim 12 further comprising an active agent and a cosmetically or pharmaceutically acceptable vehicle.

14. The composition for topical application to skin of claim 13 wherein the active agent is lipophilic.

15. The composition for topical application to skin of claim 13 wherein the active agent is water soluble or water dispersible.

16. The composition for topical application to skin of claim 13 wherein the active agent is elected from the group consisting of sunscreens, moisturizers, emollients, occlusives, antioxidants, antimicrobials, antifungals, anti-acne agents, topical analgesics, insect repellents and skin protectants.

17. The composition for topical application to skin of claim 13 wherein the active agent is selected from the group consisting of oils, C12 to C24 fatty alcohols, esters of a fatty acids and an alcohol, glyceryl esters, lipids, ceramides, nonvolative silicone fluids, lanolin, benzyl laurate, cetyl esters, dimethicone copolyol, glycerol isosterates, propylene glycol laurate, glycerin, inulin, sorbitol, fish liver oil, squalane, methicone, mineral oil, petrolatum, allantoin, aluminum acetate, aluminum hydroxide, aluminum sulfate, calamine, cocoa butter, cod liver oil, colloidal oatmeal, dimethicone, glycerin, kaolin, lanolin, mineral oil, petrolatum, shark liver oil, sodium bicarbonate, talc, witch hazel, zinc acetate, zinc carbonate and zinc oxide, and mixtures thereof.

18. The composition for topical application to skin of claim 16 wherein the active is a sunscreen.

19. The composition for topical application to skin of claim 18 wherein the sunscreen is selected from the group consisting of aminobenzoic acid, avobenzone, cinoxate, dioxybenzone, menthyl anthranilate, octocrylene, octyl methoxycinnamate, oxybenzone, padimate O, phenylbenzimidazole sulfonic acid, DEA methoxycinnamate, drometrizole trisiloxane, octyl salicylate, homomenthyl salicylate, octyl dimethyl PABA, TEA salicylate, 4-methyl benzylidene camphor, terephthalylidiene dicamphor sulfonic acid, ethyl PABA, hydroxy methylphenyl benzotriazole, methylene bis-benzotriazoyltetramethylbutylphenol, bis-ethylhexyloxyphenol methoxyphenol triazine, octyl triazone, polysilicone-15, diethylhexyl butamido triazone, diethylamino hydroxybenzoyl hexyl benzoate, and mixtures thereof.

20. A method of modifying an aesthetic characteristic of a composition for topical application, the method comprising incorporating the aesthetic modifier composition of claim 1 into said composition for topical application.
